(19) United States
(12) Plant Patent Application Publication
(10) Pub. No.: US 2015/0020247 P1
(43) Pub. Date: Jan. 15, 2015

(54) Strawberry plant named 'Deluxe'

(50) ;

(71) Applicant: Alexandre Pierron-Darbonne, Navarra (ES)

(72) Inventor: Alexandre Pierron-Darbonne, Navarra (ES)

(21) Appl. No.: 13/987,146

(22) Filed: Jul. 3, 2013

(51) Int. Cl. A01H 5/00

(52) U.S. Cl. PLT/208

(57) ABSTRACT

A new and distinct strawberry variety, Fragaria×ananassa, cv. “Deluxe” is characterized by an orange red fruit color, a fruit shape that ranges from round to conical, a medium leaf size, and a medium green color of the upper side of the leaf.

### LATIN NAME OF THE GENUS AND SPECIES CLAIMED

Fragaria×ananassa

### VARIETY DENOMINATION

‘Deluxe’

### BACKGROUND AND SUMMARY OF THE INVENTION

The present invention relates to a new and distinct strawberry variety. The varietal denomination of the new variety is “Deluxe”. The new variety was designated by the breeder as “Deluxe (DA46)”. The new variety of strawberry was created in a breeding program by crossing two parents in 2004 in Le Barp, France; in particular, by crossing as seed parent an undistributed strawberry parent designated “16.01.18” (unpatented) and as pollen parent an strawberry parent designated “Darselect” (U.S. Plant Pat. No. 10,402). Seed parent is a selection from breeder's program and has not been commercialized.

The resulting seedling of the new variety was grown and asexually propagated by runners in Le Barp, France 0.7° W., 44°, 50 meters elevation. Clones of the new variety were further asexually propagated and extensively tested. This propagation and testing has demonstrated that the combination of traits disclosed herein which characterize the new variety are fixed and retained true to type through successive generations of asexual reproduction.

Among the characteristics which appear to distinguish the new variety from its closest variety of which I am aware (which is also the pollen parent of the new variety), “Darselect” is a combination of traits which include: a medium leaf size, as compared to a large leaf size for “Darselect,” a medium green color of the upper side of the leaf, as compared to light green for “Darselect,” an orange red fruit color as compared to red for “Darselect,” and a fruit shape that ranges from round to conical, as compared to conical for “Darselect.”

On May 7, the average fruit mass (g/fruit) observed for the new variety “Deluxe” was 32.4 g, as compared to 24.5 g for “Darselect.” The luminosity for the fruit of the new variety at 460 nm was 62.5, as compared to 51.2 for “Darselect.”

Characteristics which appear to distinguish the new variety from the variety “Clery,” at least include that the new variety has an average fruit mass of 32.4 g and a luminosity at 460 nm of 62.5, as compared to an average fruit mass of 18.9 g and luminosity at 460 nm of 43.5 for “Clery.”

### BRIEF DESCRIPTION OF PHOTOGRAPHS

The accompanying photographs show typical specimens of the new variety, designated “Deluxe” or “DA 46” in the illustrations, including fruit, foliage and flower, in color as nearly true as it is reasonably possible to make in color illustrations of this character.

FIG. 1 shows several plants of the new variety “Deluxe.”

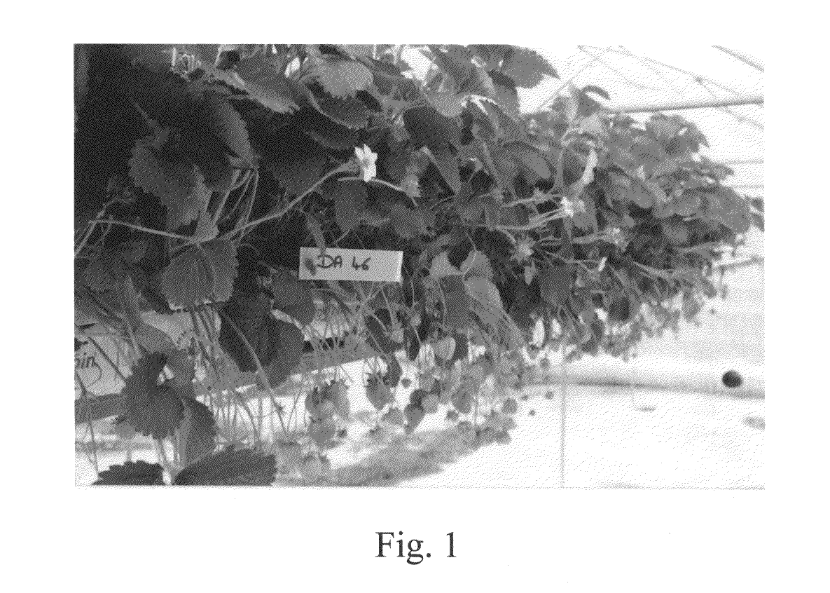

FIG. 2 shows several plants of the new variety “Deluxe.”

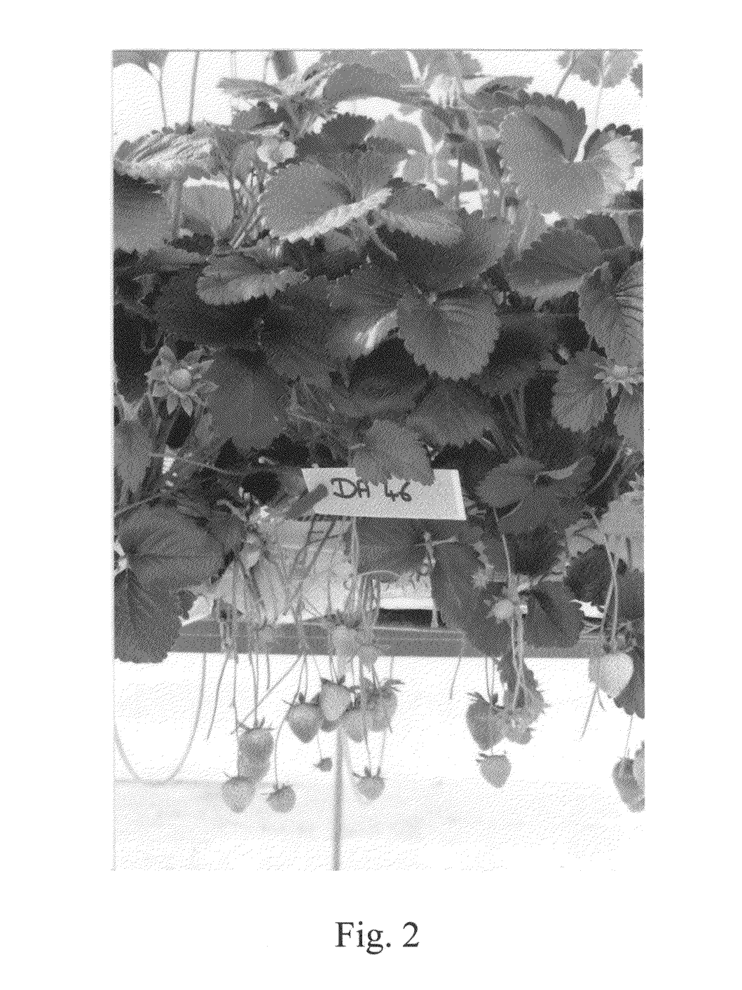

FIG. 3 shows the upper side of a complete leaf of the new variety “Deluxe.” A medium leaf size, medium green color, and strongly concave to slightly concave cross-section can be seen.

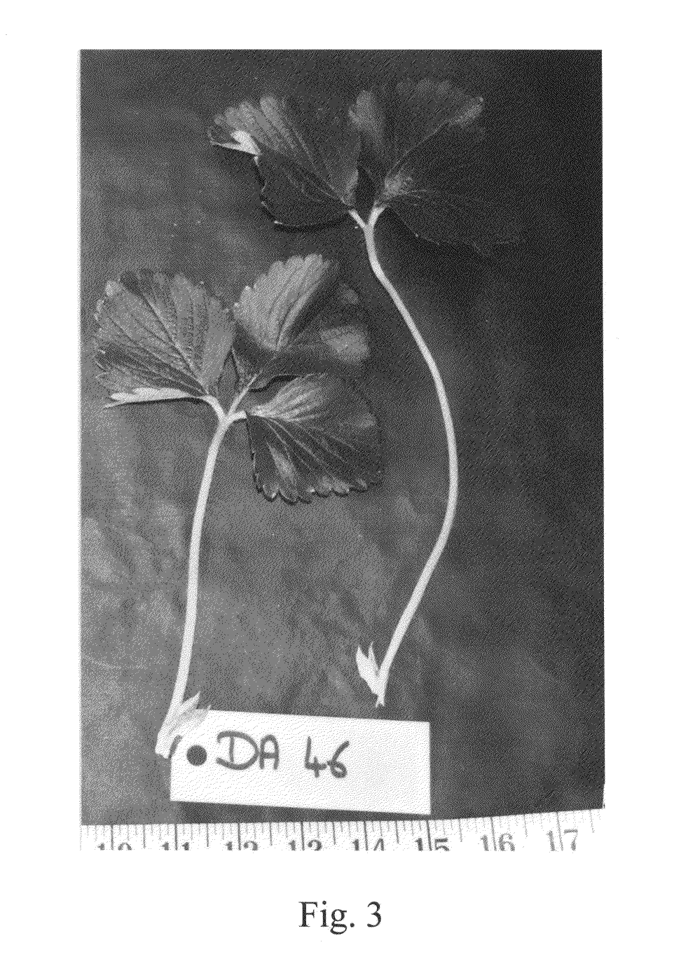

FIG. 4 shows the upper side of a terminal leaflet of the new variety “Deluxe.” A medium green color, and serrate shape of incisions of the margin of the terminal leaflet can be seen.

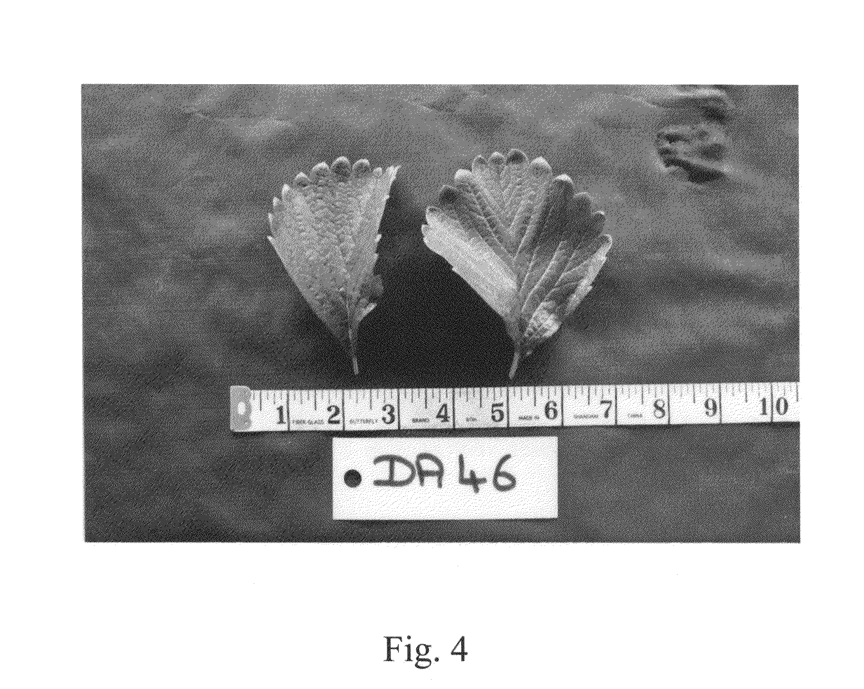

FIG. 5 shows a comparison of the upper side of a complete leaf of the new variety “Deluxe” to that of the pollen parent, “Darselect.” A medium leaf size, and medium green color can be seen in the new variety, as compared to a large leaf size and light green color for “Darselect.”

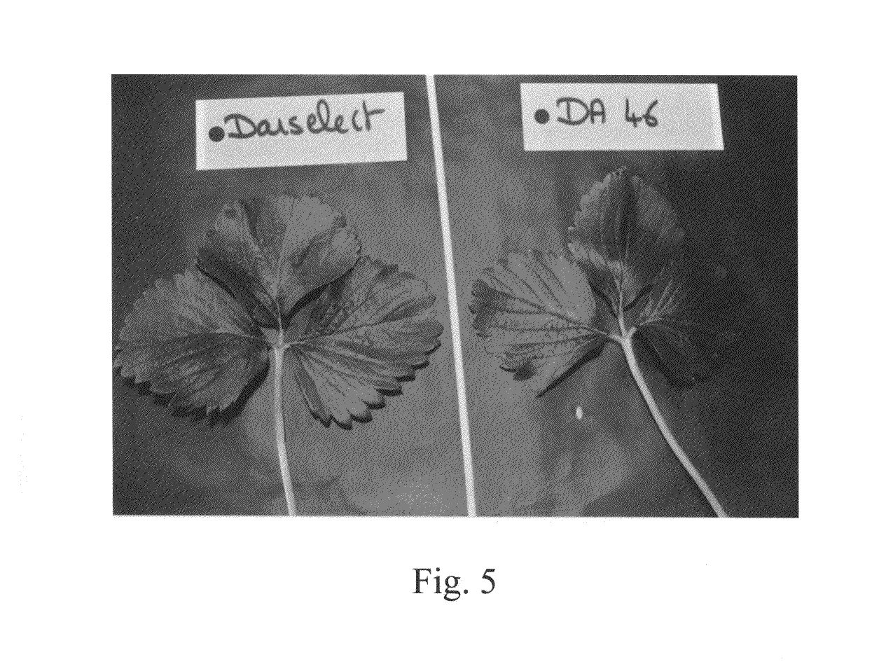

FIG. 6 shows the stipule and petiole of the new variety, “Deluxe.”

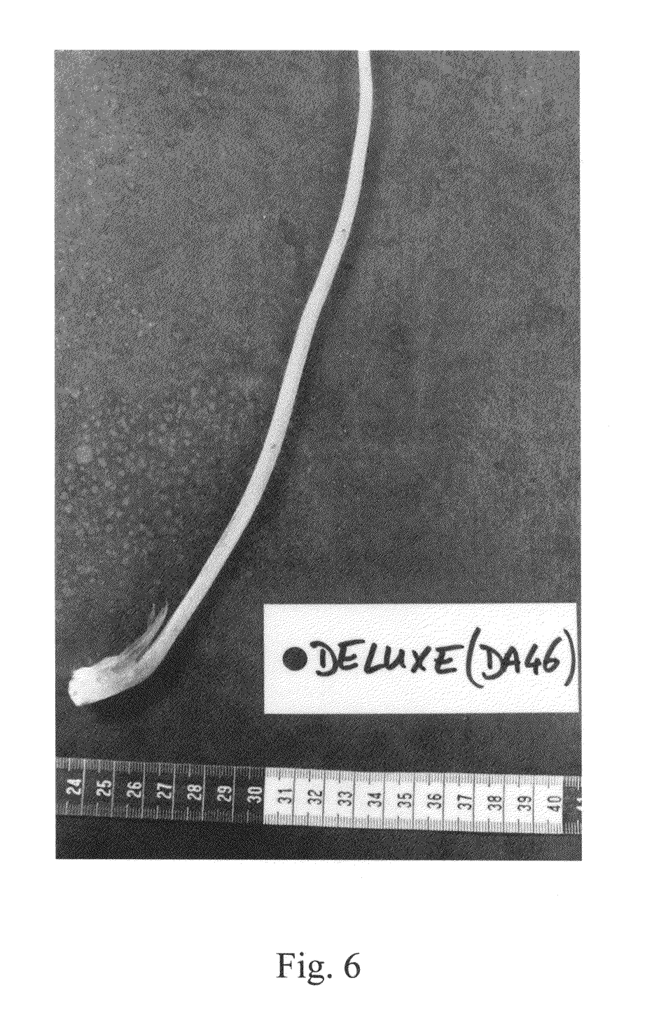

FIG. 7 shows the stipule and petiole of the variety “Darselect.”

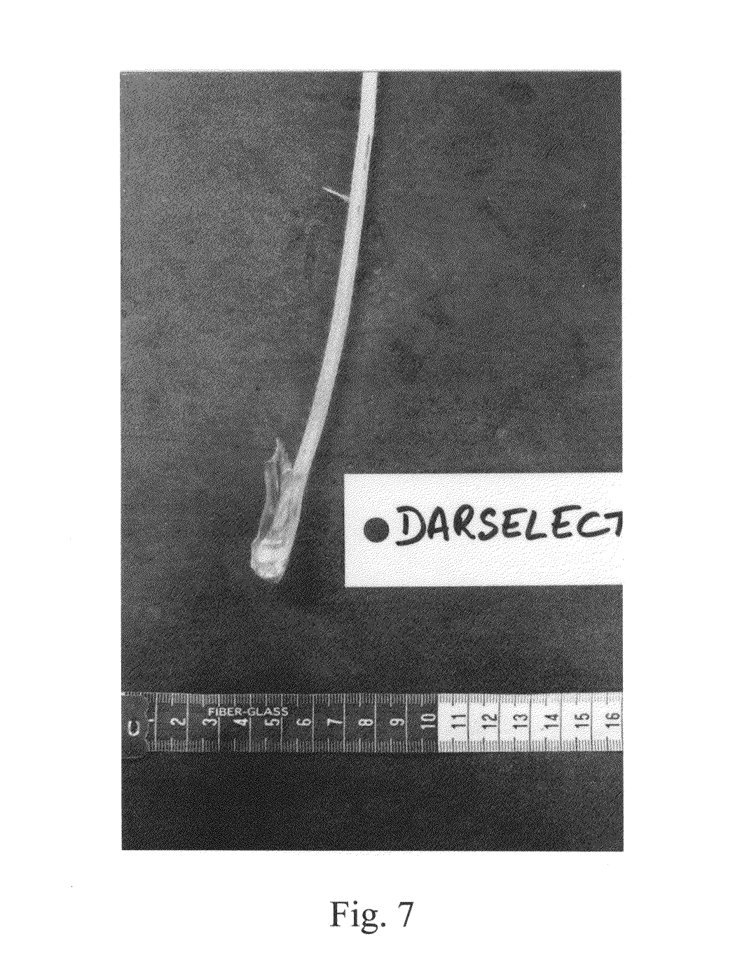

FIG. 8 shows the flower of the new variety, “Deluxe.”

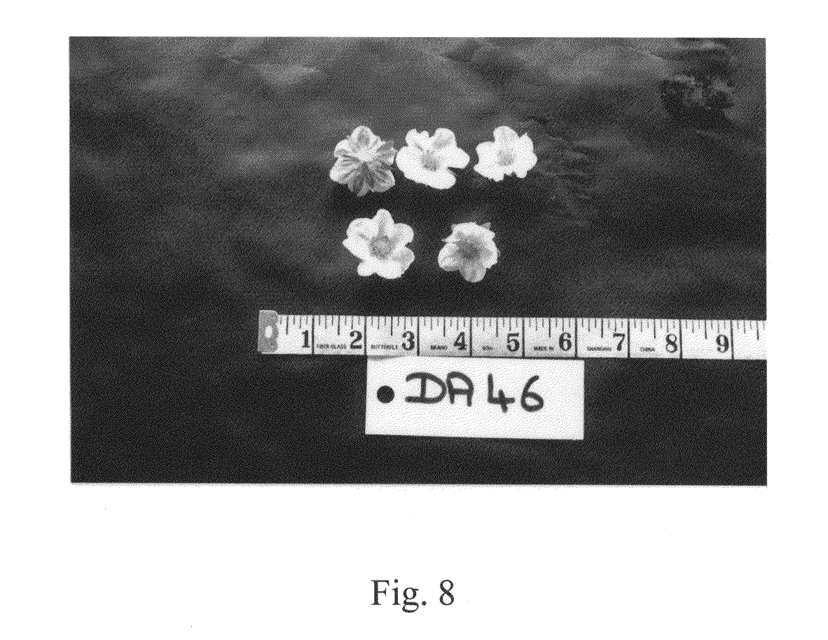

FIG. 9 shows typical whole fruit of the new variety “Deluxe,” illustrating the typical conical shape, orange red color, and strong glossiness.

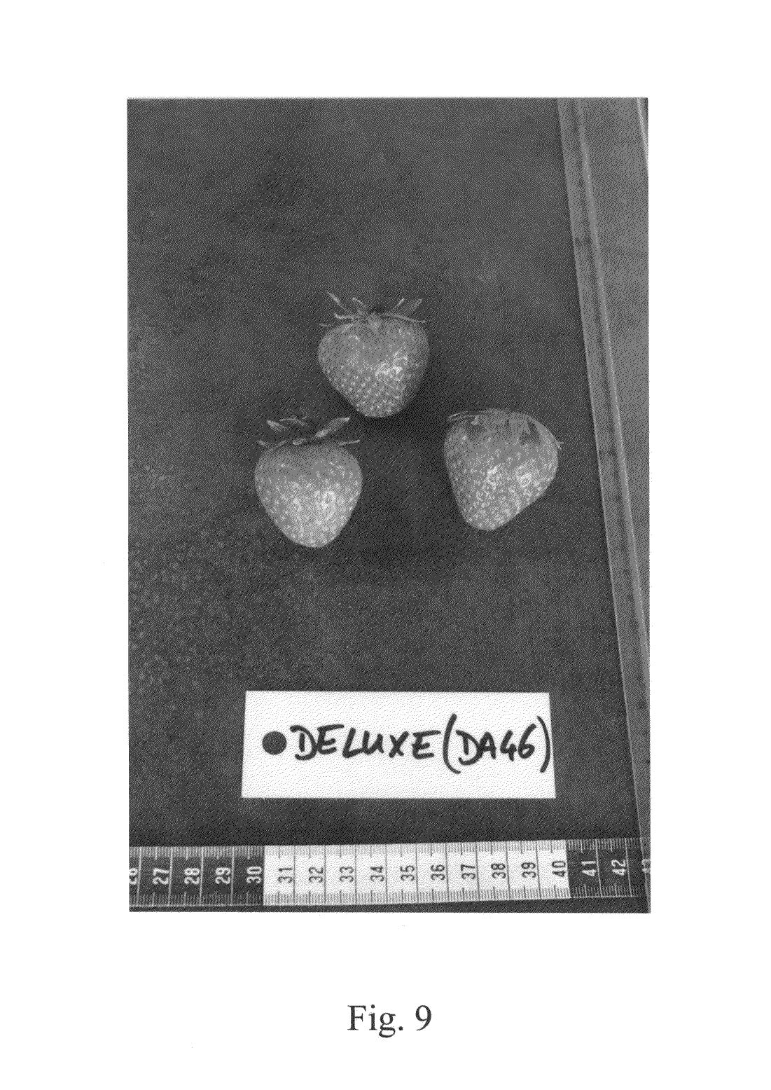

FIG. 10 shows a typical sliced section of the fruit of the new variety “Deluxe,” illustrating the typical flesh coloration of about orange red and a very weakly expressed hollow center.

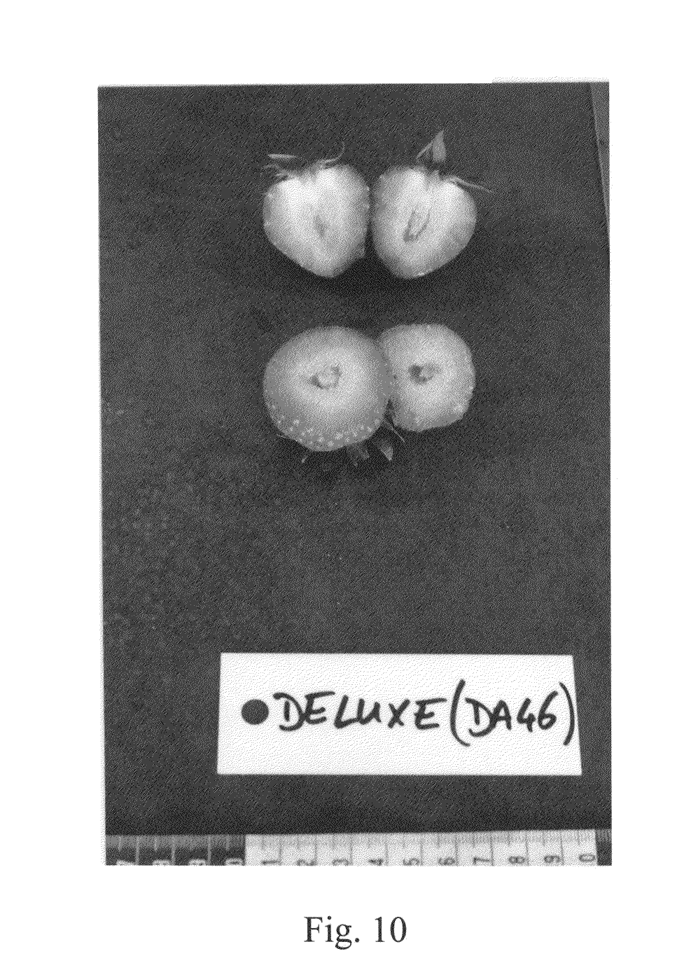

FIG. 11 shows a comparison of typical whole fruit of the new variety “Deluxe” to that of the pollen parent “Darselect,” illustrating the typical round to conical shape of the fruit of the new variety.

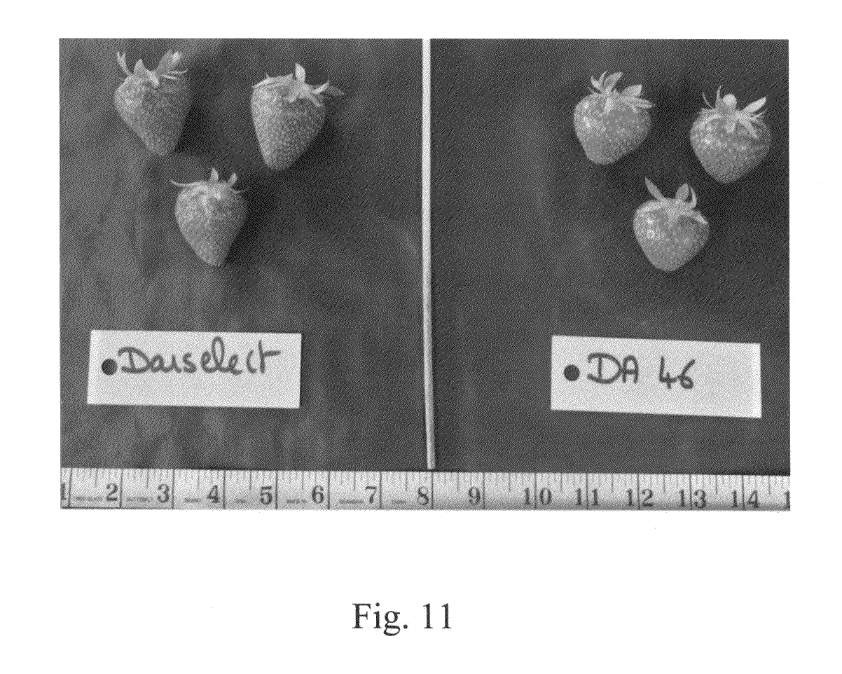

### DESCRIPTION OF THE NEW VARIETY

Throughout this specification, color names beginning with a small letter signify that the name of that color, as used in common speech is aptly descriptive. Color names beginning with a capital letter designate values based upon the R.H.S. Colour Chart published by The Royal Horticultural Society, London, England, 1995. The color descriptions and other phenotypical descriptions may deviate from the stated values and descriptions depending upon variation in environmental, seasonal, climatic and cultural conditions.

The following The following detailed description of the new variety is based upon observations taken of plants and fruits grown in Le Barp, France 0.7° W., 44°, 50 meters elevation.

The new variety is principally propagated by way of runners. Although propagation by runners is presently preferred, other known methods of propagating strawberry plants may be used. Strawberries root well after transplanting.

The term “blistering” used herein refers to the texture or rugosity or surface ondulation inherent to leaves and is generally a constant characteristic.

Table 1 shows the Weight (g/Fruit) at of the new variety “Deluxe” on May 7 when compared to its pollen parent and closest variety “Darselect” and the variety “Clery.”

TABLE 1

| WEIGHT (g/fruit)ⁱ | May 7 |
|---|---|
| Deluxe | 32.4 |
| Darselect | 24.5 |
| Clery | 18.9 |

ⁱWEIGHT is shown as the average weight per fruit in First Quality Fruits.

Table 2 shows a comparison of the fruit analysis between the new variety “Deluxe on May 7 when compared to its pollen parent and closest variety “Darselect” and the variety “Clery.”

TABLE 2

| | DELUXE | DARSELECT | CLERY |
|---|---|---|---|
| Humidity & Volatile Matter (%) | 92.7 | 91.5 | 91.5 |
| Dry Matter (%) | 7.3 | 8.5 | 8.5 |
| pH (to 20°) | 3.9 | 3.8 | 3.8 |
| Acidity as Anhydride Citric (%) | 0.6 | 0.6 | 0.6 |
| Soluble solids (°Brix) | 7.5 | 7.8 | 7.9 |
| Maturity Index | 12.5 | 13.0 | 13.2 |
| Dominant Tonality (nm) | 490 | 490 | 495 |
| Luminosity: Transmittance to 460 nm | 62.5 | 51.2 | 43.5 |

B. Dry Matter: It is the weight of the residual left from the trituration of the fruit after the drying process at a temperature of 103° C.⁺2° C. until reaching constant weight.

$$\% Dry Matter = \frac{Weight Dry Matter}{Weight Fresh Matter} \times 100$$

C. Humidity & Volatile Matter: Represents the content in volatile matters and water of the fruits.

(%) Humidity & Volatile Matter=100−% Dry Matter
D. Maturity Index: Relation between Soluble solids and Acidity as Anhydride Citric.

$$Maturity Index = \frac{Soluble solids}{Acidity as Anhydride Citric} .$$

### DETAILED DESCRIPTION OF THE NEW VARIETY

Plant:
   Habit.—Globose.
   Growth habit.—Upright.
   Density.—Dense.
   Vigor.—Strong.
Leaf:
   Size.—Medium.
   Color of upperside.—Medium Green.
   Cross section.—Strong concave to slightly concave.
   Blistering.—Weak.
   Glossiness.—Medium.
Terminal leaflet:
   Length/width ratio.—Longer than broad.
   Terminal leaflet shape of base.—Acute.
   Shape of incisions at margin.—Serrate.
Petiole:
   Attitude of hairs.—Upwards.
Stipule:
   Size.—Absent or very small.
   Anthocyanin coloration.—Absent or very weak.
Stolons:
   Number.—Medium.
   Antocyanin coloration.—Weak.
   Pubescence.—Medium.
Inflorescence:
   Position relative to foliage.—Level with.
Flower:
   Size.—Medium.
   Size of calyx relative to corolla.—Smaller.
   Primary flower relative position of petals.—Overlapping.
Petal:
   Length/width ratio.—As long as broad.
   Color.—White.
Fruit:
   Ratio of length/maximum width.—Slightly longer than broad.
   Size.—Medium.
   Fruit shape.—Concial.
   Difference in shapes between primary and secondary fruits.—Moderate.
   Band without achenes.—Absent or very narrow.
   Unevenness of surface.—Weak.
   Color.—Orange red.
   Evenness of color.—Even.
   Glossiness.—Strong.
   Insertion of achenes.—Level with surface.
   Insertion of calyx.—Level with fruit.
   Attitude of the calyx segments.—Spreading.
   Size of the calyx in relation to fruit diameter.—Same size.
   Adherence of calyx.—Strong.
   Firmness.—Firm.
   Color of flesh.—Orange red.
   Hollow center.—Absent or very weakly expressed.
   Distribution of red colour of lesh.—Marginal and central.
   Time of flowering.—Medium.
   Time of ripening.—Medium.
   Type of bearing.—Not remontant.
Disease resistance: No particular sensitivity to any disease or parasite has been observed for “Deluxe”.

What is claimed is:

1. A new and distinct strawberry plant of the variety substantially as shown and described.
